(19) United States
(12) Patent Application Publication
(10) Pub. No.: US 2022/0211356 A1
(43) Pub. Date: Jul. 7, 2022

(54) DEVICE FOR SKIN BIOPSY

(71) Applicants: Singapore Health Services PTE LTD, Singapore (SG); Singapore University of Technology and Design, Singapore (SG)

(72) Inventors: Chi Yuan Ian Loh, Singapore (SG); Yuxin Evelyn Tay, Singapore (SG); Subburaj Karupppasamy, Singapore (SG); Tomy Arden Phang, Singapore (SG); Ming Yew Sim, Singapore (SG); Benjamin Ng, Singapore (SG); Vincent Sin, Singapore (SG)

(21) Appl. No.: 17/429,923

(22) PCT Filed: Feb. 11, 2020
(86) PCT No.: PCT/SG2020/050065

§ 371 Date: Aug. 10, 2021

(30) Foreign Application Priority Data
Feb. 11, 2019 (SG) 10201901144Y

(51) Int. Cl. A61B 10/02

(52) U.S. Cl. CPC A61B 10/0266; A61B 2010/0208

(57) ABSTRACT

A skin biopsy device and a method of operating a skin biopsy device are disclosed. The skin biopsy device comprises a base member configured to be placed onto a skin surface; an cutting member configured to be received by the base member and to be movable relative to the base member in a direction substantially orthogonal to the skin surface; and an actuation member configured to drive the cutting member to travel a predetermined distance at a predetermined velocity in said direction.

### FIELD OF INVENTION

The present invention relates broadly, but not exclusively, to devices for skin biopsy.

### BACKGROUND

Skin biopsy is a biopsy technique in which a skin lesion is removed and sent to a pathologist or dermatologist to render a microscopic diagnosis. Skin biopsy not only helps in diagnosis in cases of dilemma but also provides an opportunity to find something unusual in routine practice. As skin biopsy is typically done in a setting of localised anaesthesia, a dermatologist may face the challenge of choosing the correct lesion and adapting the right technique for performing biopsy in order to ensure a good interpretation of the biopsy. It is a classical dictum in skin biopsy to choose the classical, well-formed, non-modified (by scratching or any topical application) lesion.

One of three types of skin biopsies is performed in order to evaluate the nature of the lesion. The first type of skin biopsy is the shave biopsy where a physician manually removes a thin layer of the lesion to be viewed under a microscope by a pathologist. This procedure is done using a circular punch that cuts a circle around the skin lesion and the required section of the skin is subsequently removed and stitched up. The second type of biopsy procedure is called an excision biopsy, where a physician (typically a cosmetic surgeon or a dermatologist) uses a scalpel to surgically remove the area of interest, again to be viewed by a pathologist. However, depending on the skill of the surgeon and the size of the lesion, this procedure can leave large and unsightly scars requiring multiple stitches to heal properly. The third type of biopsy is called punch biopsy, where a device with a circular cutting head is pushed into the lesion and rotated, removing a core section of the skin to be evaluated again by histology. Punch biopsy has become the preferred method of biopsy in recent times as it does not require the skill of a specialist and can be done in little time and minimal discomfort during a normal visit to the physician.

A typical circular punch biopsy device is shown in FIG. 1. The simplicity of the tubular cutting blade on a modified scalpel handle of the typical punch biopsy device makes it inexpensive to manufacture and easy to operate. However, a disadvantage of the device of FIG. 1 is that the patient is left with a more noticeable scar. By removing a circular section of skin around a lesion, closing the wound becomes difficult because there are no edges to be joined. In closing such wounds, raised edges, known as “dog-ears”, appear at either edge of the closed circle. “Dog-ears” can be defined as an upward formation of excess skin as a result of a suture. Furthermore, “dog-ears” are the result of the closure of a circular or asymmetric wound which causes surrounding skin to create pressure on the wound site and force the flaps of skin upward and outward.

Skin biopsy may also be performed during scar revision surgery. Scar revision surgery takes approximately one to three hours, depending on the scar length and complexity. During the procedure, the surgeon makes precise and small incisions of approximately 5 mm to 7 mm each while excising the scar. However, commercial blades that are currently used in scar revision are often large and unwieldly. Further, the design and incision process take a large portion of the operative time and closure of the scar and healing of the wound typically takes approximately five to seven days for scars on the face. Therefore, incisions and incision designs that are imprecise may cause unfavourable results and can delay operative time and wound healing.

Accordingly, a need exists to provide a device for skin biopsy that seeks to address some of the above problems.

### SUMMARY

According to a first aspect of the present invention, there is provided a skin biopsy device comprising: a base member configured to be placed onto a skin surface; an cutting member configured to be received by the base member and to be movable relative to the base member in a direction substantially orthogonal to the skin surface; and an actuation member configured to drive the cutting member to travel a predetermined distance at a predetermined velocity in said direction.

In an embodiment, the base member may be made of a transparent material.

In an embodiment, the base member may have an alignment element to align the cutting member with a shape of an incision on the skin surface.

In an embodiment, the base member may be movable relative to the skin surface, and wherein the base member is configured to generate a tension on the skin surface.

In an embodiment, the cutting member may be mounted to a cartridge, and wherein the cartridge is received by the base member.

In an embodiment, the cutting member may comprise a fusiform profile having a length to width ratio of about 3 to 1.

In an embodiment, the fusiform profile may comprise an apical angle of about 30 degrees.

In an embodiment, the cutting member may comprise a profile having a pair of matching zig-zag lines separated by a pre-determined gap.

In an embodiment, the cutting member may comprise a profile having at least one zig-zag line, and wherein the at least one zig-zag lines comprises a segment defining an obtuse angle.

In an embodiment, the cutting member may be made of stainless steel.

In an embodiment, the actuation member may comprise a spring-loaded mechanism.

In an embodiment, the spring-loaded mechanism may comprise a dampener.

In an embodiment, the actuation member may comprise a gas-powered mechanism.

In an embodiment, the actuation member may be configured to provide a force of about 130N.

In an embodiment, the actuation member may have a pistol and trigger form.

In an embodiment, the predetermined distance may be at least 6 mm.

According to a second aspect of the present invention, there is provided a method of operating a skin biopsy device comprising a base member, a cutting member and an actuation member, the method comprising: placing the base member onto a skin surface; disposing the cutting member in the base member such that the cutting member is movable relative to the base member in a direction substantially orthogonal to the skin surface; and operating the actuation member to drive the cutting member to travel a predetermined distance at a predetermined velocity in said direction.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1 shows a plan view of a typical circular punch biopsy device.

FIG. 2 shows a side view of a skin biopsy device, according to an example embodiment.

FIG. 3 shows a perspective view of the base member of the device of FIG. 2, according to an example embodiment.

FIG. 4 shows a perspective view of the cutting member of the device of FIG. 2, according to an example embodiment.

FIG. 5A shows a plan view of a blade end of the blade of FIG. 4 used for W-plasty procedure, according to an example embodiment.

FIG. 5B shows a plan view of a mid-blade of the blade of FIG. 4 used for W-plasty procedure, according to an example embodiment.

FIG. 5C shows a plan view of a mid-blade of the blade of FIG. 4 used for Z-plasty procedure, according to an example embodiment.

FIG. 5D shows a plan view of a blade end of the blade of FIG. 4 used for Z-plasty procedure, according to an example embodiment.

FIG. 5E shows a plan view of an alternative mid-blade used for Z-plasty procedure, according to an example embodiment.

FIG. 6 shows a perspective view of the cutting member received by the base member, according to an example embodiment.

FIG. 7A shows a plan view of the actuation member, according to an example embodiment.

FIG. 7B shows a side view of the actuation member, according to an example embodiment.

FIG. 8 shows a flow chart illustrating a method for skin biopsy, according to the example embodiments.

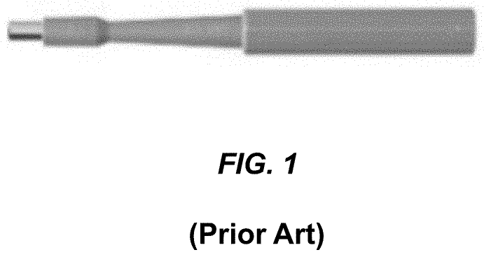

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description. Herein, devices for skin biopsy are presented in accordance with present embodiments which may have the advantages of providing a high-velocity punch and delivering momentum onto an elliptical blade to puncture an elliptical wound onto the skin, thereby using minimal manual force to penetrate the skin during operation. It may also minimise scarring during a punch biopsy process and may also produce a clean cut biopsy specimen with minimal collateral tissue injury. The device may also be used easily, can be sterilisable and reusable, thus minimising wastage.

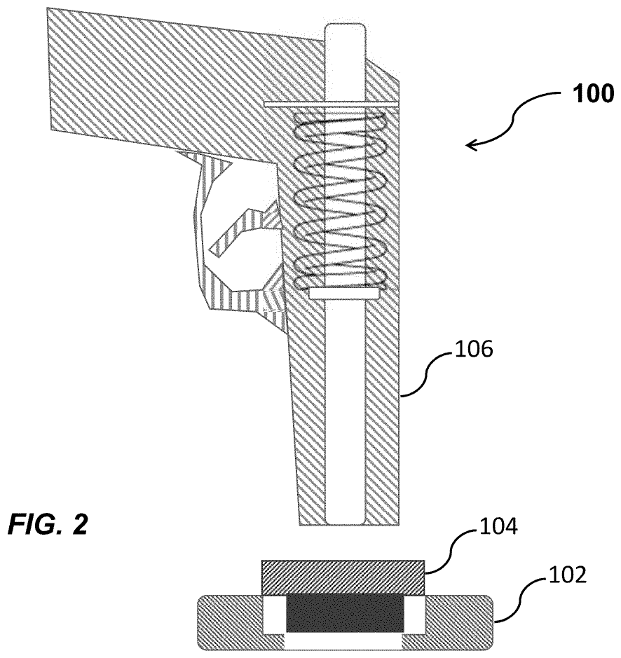

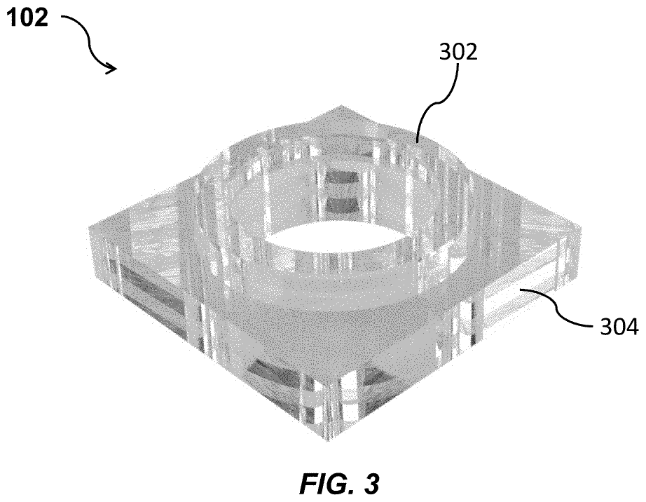

FIG. 2 shows a side view of a skin biopsy device 100, according to an example embodiment. The device 100 includes a base member 102, a cutting member 104 and an actuation member 106. The base member 102 is configured to be placed onto a skin surface while the cutting member 104 is configured to be received by the base member 102 and to be movable relative to the base member 102 in a direction substantially orthogonal to the skin surface. The actuation member 106 is configured to drive the cutting member 104 to travel a predetermined distance, e.g. 6 mm or more, at a predetermined velocity in the direction substantially orthogonal to the skin surface. A detailed view of the base member 102, the cutting member 104 and the actuation member 106 are shown in FIGS. 3-7B and will be explained in more detail below.

FIG. 3 shows a perspective view of the base member 102 of the device 100 of FIG. 2, according to an example embodiment. The base member 102 may be a jig that is made of a transparent material, e.g. polyethylene plastic, as shown in FIG. 3. By being transparent, the base member 102 may provide visual aid of the cut area such that a user of the skin biopsy device 100 may be able to see the area of cut before the cutting member 104 makes the cut using energy that is released from the actuation member 106. This may increase the accuracy of the cut as the user may be able to position the actuation member 106 accurately. The base member 102 may include a blade receiver 302 to receive the cutting member 104. The base member 102 may also include a support 304 such that a bottom surface of the support 304 is placed on the skin surface.

The base member 102 may have an alignment element (not shown) to align the cutting member 104 with a shape of an incision on the skin surface. An example of an alignment element may be a collagen line aligner. The base member 102 may be movable relative to the skin surface and may also be configured to generate a tension on the skin surface. The base member 102 may hold the cutting member 104 in position while the actuation member 106 (e.g. a gun) is aimed at the base member 102 to release an actuation momentum to puncture into the skin. Further, the base member 102 may also work as a depth-control tool such that the cutting member 104 does not penetrate too deep into the skin when making the incision.

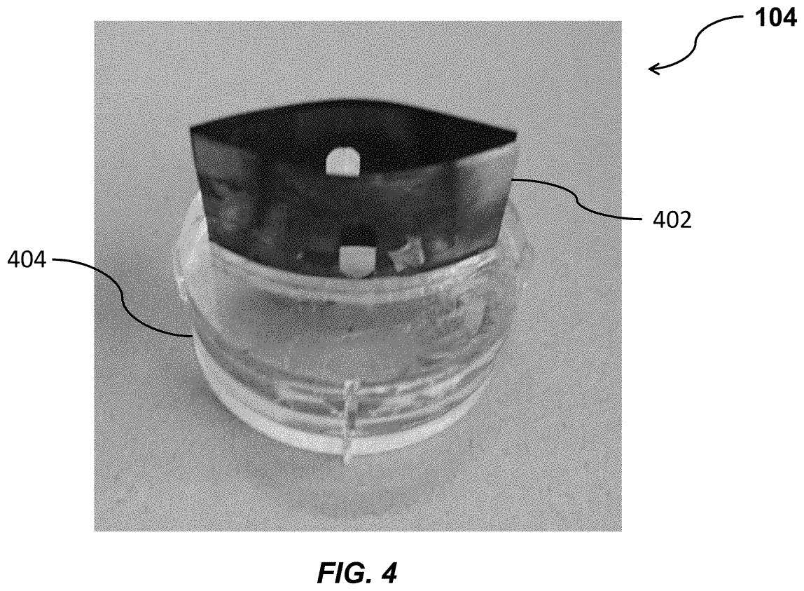

FIG. 4 shows a perspective view of the cutting member 104 of the device 100 of FIG. 2, according to an example embodiment. The cutting member 104 may include a blade 402 having a straight and pointed blade profile which can provide a clean and sharp cut. The straight and pointed blade profile may reduce scarring associated with traditional punch biopsy as the incision made by the straight and pointed blade profile can have two even edges which can meet to form a straight line during stitching of the wound.

Healing of a closed wound is affected by factors such as size and geometry of incision and the distribution and magnitude of the stresses on the wound. The circular wound has the worst healing, with maximum stresses ranging from 40% to 62% greater than the other shapes. The elliptical excisions have the least adverse maximum stresses, while the fusiform excision has minimum stress. High adverse stresses adversely affect the microcirculation in the area around the wound, slowing the healing of the wound. It was found that aligning the incision along Langer lines (i.e. topological lines on a human body) results in lower stresses when closing the wound, thereby assisting in the healing and reducing scarring of the skin. In addition, surgical techniques can play a role in “dog-ear” formation. For example, improper surgical techniques can cause excess tissue to be present at the wound site, which may occur due to the tendency of surgeons deferring from the 90° proper cutting angle. According to an embodiment, the blade 402 may also be of a fusiform profile having a length to width ratio of approximately 3:1. The fusiform profile may have an apical angle of about 30 degrees.

The blade 402 may be made of stainless steel. In one embodiment, the stainless steel blade 402 may be made using 304 stainless steel. For example, to make the blade, plates measuring 2.64 cm in length and 4 cm in width are cut out from a larger plate using a water jet. The edges of the blade 402 are initially grinded to a preliminary bevel. A Lansky 5 stone deluxe sharpening system is subsequently used for finer grit sharpening. Various grits may be used, starting from 70, 120, 280, 600 to 1000 grit. After reaching 1000 grit, different stones can be used to improve sharpness and surface finish of the blade 402. In an alternative embodiment, the blade 402 may be made of high carbon steel. The high carbon steel blade 402 is hard and may be able to hold a grinded edge for a longer period of time than normal steel blades. This may result in sharper blades that do not dull after a single use. The high carbon steel blade 402 may be sharpened until it is able to push cut paper and then bent into an elliptical shape. It can also be appreciated that alternative manufacturing and bending methods can provide blades that can maintain the sharp edge, achieve precise geometry and do not rust. In an example implementation, heat treatment on the blade 402 may be carried out using a blowtorch. More specifically, the blade 402 is heated up, bent, cooled and the process is repeated. Such a method of heat treatment may allow the blade's edge to be maintained longer. The cutting member 104 may include a blade holder 404 made up of multiple (e.g. four) layers of clear acrylic in which the blade 402 is mounted. The acrylic pieces of the blade holder 404 are cut with slots that provide a compression fit for the blade 402. The clear acrylic blade holder 404 may also act as a viewing window for the user. In addition, the blade holder 404 is dimensioned to fit into the blade receiver 302 of the base member 102.

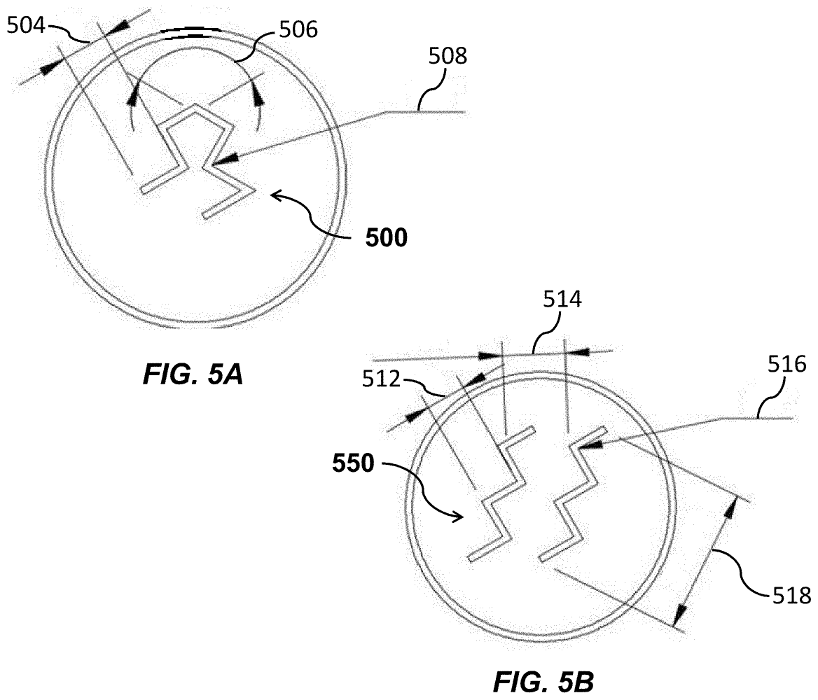

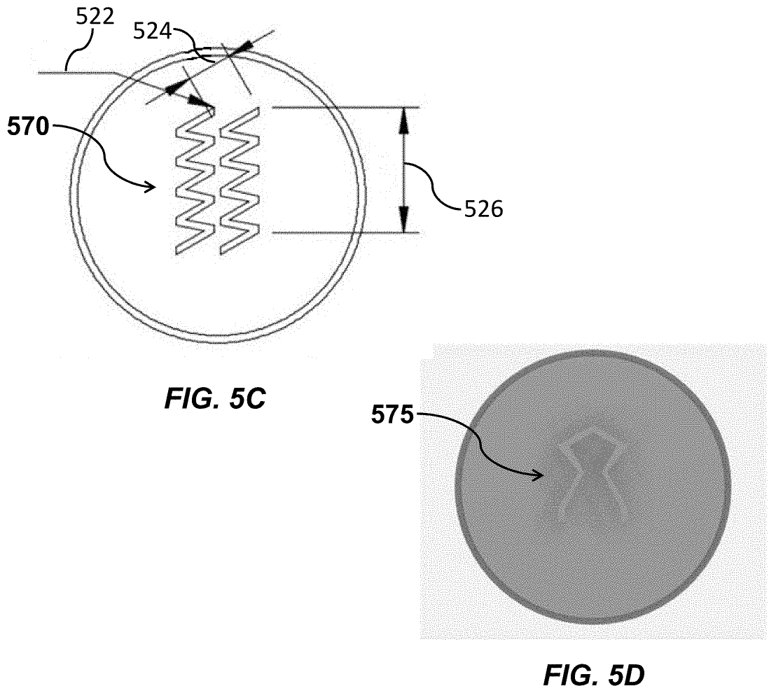

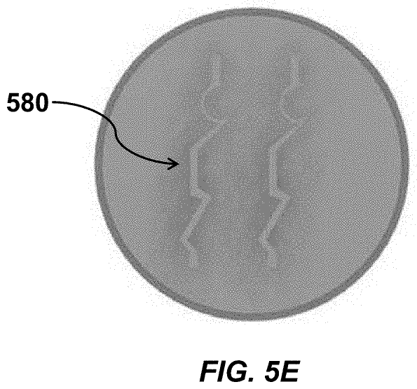

FIG. 5A shows a plan view of a blade end 500 of the blade of FIG. 4 while FIG. 5B shows a plan view of a mid-blade 550 of the blade of FIG. 4 used in a W-plasty procedure, according to an example embodiment. The blade as shown in the Figures can be used in the W-plasty technique of scar revision (or scar healing). In an embodiment, the blade may consist of a zigzag, geometric broken line with the blade end 500 having dimensions of 6 mm, 120° and 60°, representing numerals 504, 506 and 508 respectively in FIG. 5A. The mid-blade 550 may have dimensions of 6 mm, 9.24 mm, 60° and 21.63 mm, representing numerals 512, 514, 516 and 518 respectively in FIG. 5B. In an embodiment, the blade may include a profile having a pair of matching zig-zag lines separated by a pre-determined gap. The blade may also include a profile having a pair of zig-zag lines joining at their respective ends to form an obtuse angle.

In an alternate embodiment, the blade may be used for Z-plasty procedure. In this example, the blade may have mid-blade 570 dimensions of 60°, 6 mm and 17 mm, representing numerals 522, 524 and 526 respectively as shown in FIG. 5C. A blade end 575 of the blade used for Z-plasty procedure is shown in FIG. 5D while an alternative mid-blade 580 can be used for Z-plasty procedure is shown in FIG. 5E. It will be appreciated that the dimensions and number of zig-zag lines forming the blades as described with reference to FIGS. 5A-5E may vary in alternate embodiments.

The blade profiles as described with reference to FIGS. 5A-5E can be selectively used in a scar revision (or scar healing) surgery to improve or reduce the appearance of scars. The following techniques are commonly used during the formation of unfavourable scars in various locations on the body, particularly the face, due to its conspicuous location. Scar revision can be performed using W-plasty technique or geometric broken line closure, which is a surgical procedure to irregularize an unfavourable scar for cosmesis. Irregularizing a linear scar causes light to scatter in an irregular manner and shortens the limbs of individual limbs of the scar to less than 7 mm, making it less conspicuous during examination by the human eye. Z-plasty technique may also be used to perform scar revision, which lengthens a contracted (shortened) scar in addition to irregularizing it. The Z-plasty technique can be used to release and lengthen scars that are causing distortion and restriction of function to surrounding structures. It can also improve scar appearance through irregularization.

The blade having the dimensions and designs in embodiments shown in FIGS. 5A-5E may economize the surgical process by allowing the design and excision of the scar to take place with a single cut of the device 100. The blade may be used in a sequential fashion, such that one blade is used to cut the ends of the design and one blade is used for the body. The body-cutting blade can be used serially to lengthen the design in order to suit the length of the scar. The blade may also allow for excision of the scar and is held in place by the base member 102. The base member 102 may allow for accurate placement of the blade while limiting the depth of the cut at the same time. The actuation device 106 may then deliver an instantaneous driving force to allow the blade to cut the skin cleanly. This may save operative time used for the design and incision phase of the surgery, and may remove human error in the design and incision portion of the surgery. The base of the wound is cut with tissue scissors and the scar excised. The wound can then be closed by using routine methods.

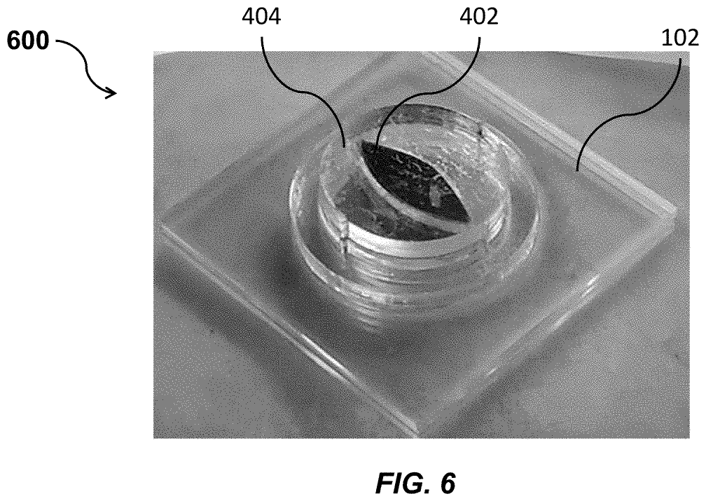

FIG. 6 shows a perspective view 600 of the cutting member 104 received by the base member 102, according to an example embodiment. The blade 402 may be mounted to the blade holder 404 such that it snugly fits into the blade receiver 302 of the base member 102. The transparent material of the blade holder 404 and the base member 102 may provide visual aid to the user before firing the actuation device 106. This may result in greater accuracy of the cut during the incision process.

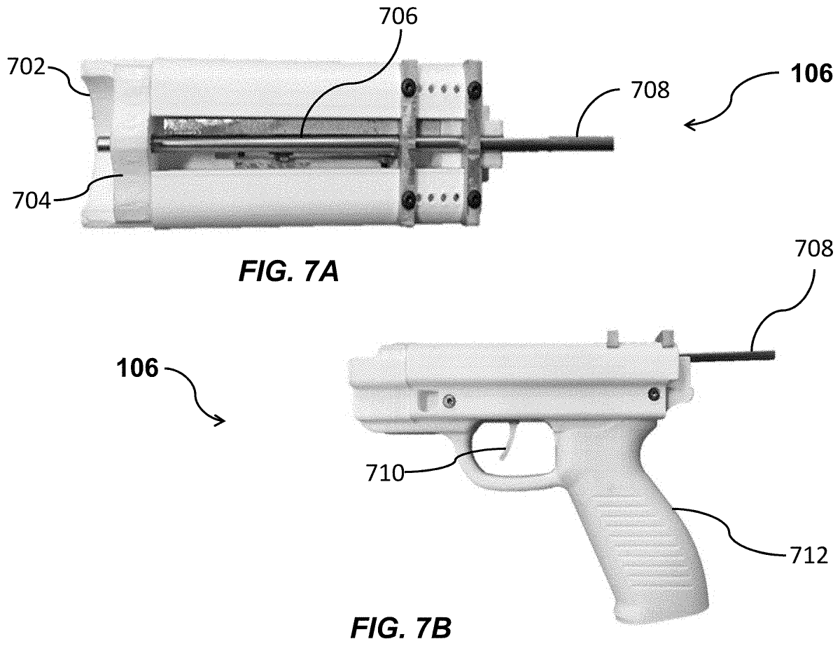

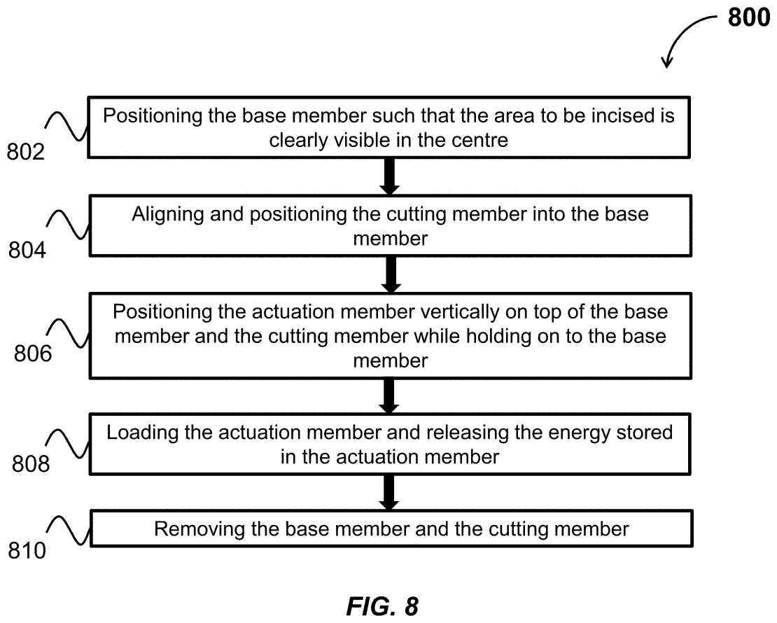

FIG. 7A shows a plan view of the actuation member 106 while FIG. 7B shows a side view of the actuation member, according to an example embodiment. As shown in the Figures, the actuation member 106 may be a pistol and trigger form, such as a gun. The actuation member 106 may include a linear guide 702, a viewing window 704 for visual aid, a trigger 710 and a handle grip 712. The actuation member 106 may include a spring-loaded mechanism, including a dampener (not shown), a hammer rod 706 and/or a linear cocking rod 708. The spring-loaded mechanism may provide energy storage before releasing the cutting member 104 as springs are more cost efficient in achieving functional requirements for punch biopsy. The spring-loaded mechanism may also have a lower margin of error, does not require air-tight design and removes the need to reload the punch with an external fluid.

A lock-and-load mechanism may be used for the release of the cutting member 104. This may have the advantage of a controlled release by the user as punch biopsy requires only a single attempt. The cocking of the spring only requires the hammer rod 706 to be pushed back into a catch (not shown) of the actuation member 106. The handle grip 712 may be ergonomic to the user which may result in better aiming and higher accuracy of the punch. Moreover, the trigger 710 can be positioned such that minimal force is required to depress the trigger 710.

In an alternate embodiment, the actuation member 106 may include a gas-powered mechanism instead of a spring for energy storage, which may make it easier for users without the need to load. The spring-loaded mechanism and/or the gas-powered mechanism may be configured to provide a force of about 130N. The energy storage can also be a hydraulic piston and/or a pressured air mechanism. Release of the cutting member 104 may be a continuous motion mechanism, which includes a single action to load and release; and/or a direct release mechanism, which includes loading the punch and release. In alternative embodiments, the handle grip 712 can be a palm down grip, a pencil grip or an ice pick grip.

In other embodiments, the interface between the hammer rod 706 and the cartridge holding the cutting member 104 can also be improved by increasing the surface area and dampening the impact slightly to remove the relatively loud sound of the hammer rod 706 hitting the cartridge, while also improving the force distribution onto the cartridge to increase the accuracy of firing.

There are different methods for skin biopsy. One method for skin biopsy is a mounted cut, which includes releasing a barrel-mounted blade onto the skin surface. Such a method may provide the advantage of having a moderate accuracy of aim and a single-hand operation by the user. Another method for skin biopsy is an external cut, where an externally-placed blade is used. The external cut method may provide a high accuracy of aim as the area to be cut is visual to the user. A third method for skin biopsy is a direct cut, where the blade is directly used to cut the skin. This may provide the advantage of having a simple design of the device used in such a method. In a preferred embodiment, the external cut configuration is used. This can be implemented in the form of a jig (or base member 102) and blade cartridge (or cutting member 104).

The typical method for the external cut is to apply the jig on the area to be cut, insert the blade, and shoot it with the gun. Depth control of the external cut can be achieved by constraining the geometry of the front barrel of the gun and the height of the jig and blade. Further, having a rotational degree of freedom while the jig is being pressed down onto the patient's skin, the oval blade can be rotated along with the jig to align the apical angles with the skin collagen lines. This may further improve scar revision (or scar healing) at the later stage.

FIG. 8 shows a flow chart 800 illustrating a method for skin biopsy, according to the example embodiments. At step 802, the method includes positioning the base member 102 such that the area to be incised is clearly visible in the centre. At step 804, the method includes aligning and positioning the cutting member 104 into the base member 102. The user may also ensure that the cutting member 104 is properly aligned by looking through the acrylic piece of the base member 102. At step 806, the method includes positioning the actuation member 106 vertically on top of the base member 102 and the cutting member 104 while holding on to the base member 102. The user may ensure a constant downward force on the base member 102 such that there is constant tension on the skin, which may allow for an even, accurate cut. At step 808, the method includes loading the actuation member 106 and releasing the energy stored in the actuation member 106, which can be performed by one person. The cutting member 104 may move a distance of at least 6 mm in the above step. At step 810, the method includes removing the base member 102 and the cutting member 104. The user can then cut out the skin specimen as required using a forcep and a scalpel and remove the specimen to proceed with suture of the wound.

In an alternative embodiment, a method of skin biopsy is described as follows. At the first step, the method includes positioning the base member 102 such that the area to be incised is clearly visible in the centre. At the second step, the method includes aligning and positioning the cutting member 104 into the base member 102, while ensuring that the cutting member 104 is properly aligned by looking through visual aids in the base member 102. At the third step, the method includes holding on to the base member 102 to ensure there is a constant downward force on the base member 102 so that there is constant tension on the skin. This may allow for an even, accurate cut. At the fourth step, the method includes positioning the actuation member 106 vertically on top of the base member 102 and the cutting member 104. At the fifth step, the method includes releasing the trigger of the actuation member 106 to release the energy stored. This step can be achieved by a single person and the cutting member 104 may move a distance of at least 6 mm in the above step. At the sixth step, the method includes removing the base member 102 and the cutting member 104. At the seventh step, the method includes using a forceps and a scalpel/or scissors to complete the cut on the deep surface of the specimen. At the eighth step, the method incudes removing the specimen and proceeding with suture.

The device for skin biopsy as described herein may be used to excise suspicious growths on the skin surface. The device may also be able to create a 30° elliptical corner at the edge of the biopsy allowing for direct edge-to-edge closure and minimise the causation of “dog-ears”. The device may be manufactured easily, cost effective, sterilisable, disposable, and ergonomically sound and easy to use. Embodiments of the present invention may provide a high velocity punch that is able to consistently produce an elliptical cut of ratio 1:3 on the skin. In addition, the sutured extracted skin area shows less “dog-ears” formation using the above high-velocity cut concept.

While exemplary embodiments have been presented in the foregoing detailed description of the invention, it should be appreciated that a vast number of variations exist.

It should further be appreciated that the exemplary embodiments are only examples, and are not intended to limit the scope, applicability, operation, or configuration of the invention in any way. Rather, the foregoing detailed description will provide those skilled in the art with a convenient road map for implementing an exemplary embodiment of the invention, it being understood that various changes may be made in the function and arrangement of elements and method of operation described in an exemplary embodiment without departing from the scope of the invention as set forth in the appended claims.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

1. A skin biopsy device comprising:
a base member configured to be placed onto a skin surface;
an cutting member configured to be received by the base member and to be movable relative to the base member in a direction substantially orthogonal to the skin surface; and
an actuation member configured to drive the cutting member to travel a predetermined distance at a predetermined velocity in said direction.

2. The device of claim 1, wherein the base member is made of a transparent material.

3. The device of claim 1, wherein the base member has an alignment element to align the cutting member with a shape of an incision on the skin surface.

4. The device of claim 1, wherein the base member is movable relative to the skin surface, and wherein the base member is configured to generate a tension on the skin surface.

5. The device of claim 1, wherein the cutting member is mounted to a cartridge, and wherein the cartridge is received by the base member.

6. The device of claim 1, wherein the cutting member comprises a fusiform profile having a length to width ratio of about 3 to 1.

7. The device of claim 6, wherein the fusiform profile comprises an apical angle of about 30 degrees.

8. The device of claim 1, wherein the cutting member comprises a profile having a pair of matching zig-zag lines separated by a pre-determined gap.

9. The device of claim 1, wherein the cutting member comprises a profile having at least one zig-zag line, and wherein the at least one zig-zag lines comprises a segment defining an obtuse angle.

10. The device of claim 1, wherein the cutting member is made of stainless steel.

11. The device of claim 1, wherein the actuation member comprises a spring-loaded mechanism.

12. The device of claim 11, wherein the spring-loaded mechanism comprises a dampener.

13. The device of claim 1, wherein the actuation member comprises a gas-powered mechanism.

14. The device of claim 1, wherein the actuation member is configured to provide a force of about 130N.

15. The device of claim 1, wherein the actuation member has a pistol and trigger form.

16. The device of claim 1, wherein the predetermined distance is at least 6 mm.

17. A method of operating a skin biopsy device comprising a base member, a cutting member and an actuation member, the method comprising:
placing the base member onto a skin surface;
disposing the cutting member in the base member such that the cutting member is movable relative to the base member in a direction substantially orthogonal to the skin surface; and
operating the actuation member to drive the cutting member to travel a predetermined distance at a predetermined velocity in said direction.
